(19) United States
(12) Patent Application Publication
(10) Pub. No.: US 2023/0105160 A1
(43) Pub. Date: Apr. 6, 2023

(54) GAS FERMENTATION CONVERSION OF CARBON DIOXIDE INTO PRODUCTS

(71) Applicant: LanzaTech, Inc., Skokie, IL (US)

(72) Inventors: Richard Russell Rosin, Glencoe, IL (US); Yen-Lu Yu, Buffalo Grove, IL (US); Sean Alex Rollag, Chicago, IL (US); Taylor Craig Schulz, Chicago, IL (US); Robert John Conrado, Washington, DC (US)

(21) Appl. No.: 17/934,664

(22) Filed: Sep. 23, 2022

Related U.S. Application Data
(60) Provisional application No. 63/251,681, filed on Oct. 3, 2021.

(51) Int. Cl. C12M 1/107; C12P 7/06; C12P 7/08; C12P 7/16; C12P 7/28; C12P 7/54; C12P 7/64; C12N 1/20

(52) U.S. Cl. CPC C12M 21/04; C12P 7/065; C12P 7/08; C12P 7/16; C12P 7/28; C12P 7/54; C12P 7/64; C12N 1/205

(57) ABSTRACT

An integrated process and system for employing low conversion rWGS to prepare a gas fermentation feed stream from a CO₂ source and a hydrogen source in order to produce at least one gas fermentation product. Low conversion rWGS reactors may (1) employ a wider selection of inorganic catalysts then rWGS reactors requiring high temperature operation, (2) allow for use of an electric heater instead of a fired heater to preheat feed stream to the low conversion rWGS reactor, and (3) extend rWGS catalyst life by reducing the amount of water produced in the rWGS reaction.

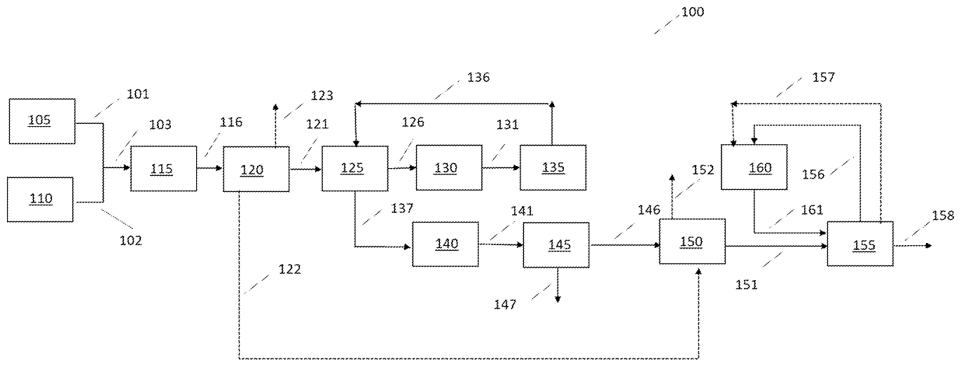

### CROSS REFERENCE TO RELATED APPLICATIONS

This application claims the benefit of U.S. Provisional Patent Application No. 63/251,681, filed Oct. 3, 2021, the entirety of which is incorporated herein by reference.

### FIELD

This application relates to an integrated process using gas fermentation to convert CO₂ into products, where low conversion reverse water gas shift is employed to convert from 20% to 60% of the CO₂ into CO before passing to the gas fermentation bioreactor(s).

### BACKGROUND

Mitigation of impending climate change requires drastic reductions in emissions of greenhouse gases (GHGs), such as those generated through the burning of fossil fuels like coal and oil. Sustainable sources of fuels and chemicals are currently insufficient to significantly displace dependence on fossil carbon. Gas fermentation is emerging to fill that gap as an alternative platform for the biological fixation of such gases such as CO, CO₂, and/or H₂ into sustainable fuels and chemicals. In particular, gas fermentation technology can utilize a wide range of feedstocks including gasified carbon-containing matter such as municipal solid waste or agricultural waste, or industrial waste gases such as off-gases from steel manufacturing, petroleum refineries, and petrochemical processes to produce ethanol, aviation fuel, chemicals, and a variety of other products. Gas fermentation alone could displace 30% of crude oil use and reduce global CO₂ emissions by 10%. As with any disruptive technology, many technical challenges must be overcome before this potential is fully achieved.

### SUMMARY

A process for producing at least one gas fermentation product from a CO₂ source and a hydrogen source is provided. The process comprises passing a combination feed stream comprising CO₂ and hydrogen to a preheater wherein the combination feed stream comprises a first feed stream comprising CO₂ and a second feed stream comprising hydrogen. The combination feed stream is heat exchanged with a reverse water gas shift reactor effluent stream to partially heat the combination feed stream and partially cool the reverse water gas shift reactor effluent stream. The partially heated combination feed stream is passed to an electric heater to fully heat the combination feed stream to a predetermined inlet temperature of a low conversion reverse water gas shift reactor. The fully heated combination feed stream is passed to the low conversion reverse water gas shift reactor to generate the reverse water gas shift reactor effluent stream comprising CO, CO₂, hydrogen, and water, wherein the conversion of CO₂ to CO in the low conversion reverse water gas shift reactor is from about 20 to about 60 mass percent. The partially cooled reverse water gas shift reactor effluent stream is passed to a cooler to generate a fully cooled reverse water gas shift reactor effluent stream. Water is removed from the fully cooled reverse water gas shift reactor effluent stream and at least one biocatalyst inhibitor is also removed to generate a syngas feed stream. The syngas feed stream is passed to a gas fermentation process to generate a gas fermentation product stream and a gas fermentation off gas stream comprising unreacted reactants selected from CO, CO₂, and hydrogen. The the gas fermentation off gas stream may be recycled to the gas fermentation process. The low conversion reverse water gas shift reactor may be operated at a temperature less than about 600° C., less than about 500° C., less than about 400° C., less than about 350° C., less than about 310° C. or less than about 300° C. The low conversion reverse water gas shift reactor may employ an inorganic catalyst comprising copper or copper oxide. The process may further comprise compressing the first feed stream comprising CO₂, the second feed stream comprising hydrogen, and or the combination feed stream comprising CO₂ and hydrogen. The process may further comprise removing one or more contaminates from the first feed stream comprising CO₂, the second feed stream comprising hydrogen, and or the combination feed stream comprising CO₂ and hydrogen. The syngas feed stream may be at a pressure ranging from about 5 to about 8 barg. The syngas feed stream may be at a pressure ranging from about 3 to about 4 barg. The syngas feed stream may have molar ratio of H₂:CO:CO₂ of about 5:1:1. The combination feed stream may comprise a molar ratio of H₂:CO₂ of from about 3:1. The syngas feed stream may comprise a molar ratio of H₂:CO:CO₂ of about 5:1:1 and the combination feed stream may comprise a molar ratio of H₂:CO₂ of from about 3:1. The low conversion reverse water gas shift reactor may be a fixed bed adiabatic reactor. The fixed bed adiabatic may comprises a single bed of catalyst. The low conversion reverse water gas shift reactor may be operated in a once through mode of operation with no interstage heating. The gas fermentation product stream may be passed to a downstream processing system to generate another product. The downstream processing system is selected from an olefins plant, a poly-olefins plant, a polypropylene plant, a polyethylene plant, a polymer plant, a high-density polyethylene plant, an oligomers plant, a nitriles plant, an oxides plant, a topping refinery, a hydro-skimming refinery, a conversion refinery, a deep conversion refinery, a coking unit, a stream cracker, or a styrenics plant. The gas fermentation process may be a microbial fermentation process employing a C1-fixing microorganism in a nutrient solution The C1 fixing microorganism may be aerobic or anaerobic. The C1-fixing microorganism may be selected from a genus of Clostridium, Moorella, Carboxydothermus, Ruminococcus, Acetobacterium, Eubacterium, Butyribacterium, Oxobacter, Methanosarcina, Methanosarcina, Desulfotomaculum, and Cupriavidus. The gas fermentation product stream may comprises at least one product selected from ethylene, ethanol, propane, acetate, 1-butanol, butyrate, 2,3-butanediol, lactate, butene, butadiene, methyl ethyl ketone (2-butanone), acetone, isopropanol, a lipid, 3-hydroxypropionate (3-HP), a terpene, isoprene, a fatty acid, 2-butanol, 1,2-propanediol, 1propanol, 1hexanol, 1octanol, chorismate-derived products, 3hydroxybutyrate, 1,3butanediol, 2-hydroxyisobutyrate or 2-hydroxyisobutyric acid, isobutylene, adipic acid, 1,3hexanediol, 3-methyl-2-butanol, 2-buten-1-ol, isovalerate, isoamyl alcohol, and monoethylene glycol, or any combination thereof.

### BRIEF DESCRIPTION OF THE DRAWING

The FIGURE is a schematic representation of one embodiment of the disclosure.

### DETAILED DESCRIPTION

The process of the disclosure provides multiple advantages, both technical and economic, due to the different requirements of the gas stream produced by a reverse water gas shift process (rWGS) when employed in the service of a gas fermentation process as compared to the more traditional use of service with a Fischer Tropsch process or a methanol synthesis process. Generally speaking, the rWGS process is employed for the purpose of converting CO₂ to CO. Although it can be said that with the water gas shift reaction being an equilibrium reaction, there is no “forward” or “reverse” reaction, in industry, the term “water gas shift” is ordinarily used to refer to the reaction of CO and water to form CO₂ and hydrogen, as this the more common desire of the reaction as used industry today. Therefore, the terms “reverse” water gas shift has taken hold to refer to the desire to react CO₂ and hydrogen to form CO and water. CO₂ is plentiful, inexpensive, and there is a need to (1) utilize CO₂ instead of emitting it into our atmosphere and (2) reduce the existing amount of CO₂ in our atmosphere already. As the rWGS process is a catalytic process to perform the water gas shift reaction of the reversible hydrogenation of CO₂ to produce CO and H₂O, the rWGS process is an upstream option for processes which require CO. Employing rWGS allows for the required CO to be generated from inexpensive, plentiful, and often waste CO₂.

Today, rWGS is commonly employed in the service of a Fischer Tropsch process which converts CO and hydrogen into hydrocarbons of various molecular weights, and in the service of a methanol synthesis processes which converts CO and hydrogen to methanol. In those services, the required conversion of CO₂ to CO in the rWGS process is typically greater than 75% and, in some cases, greater than 90%. Therefore, the synthesis gas, or “syngas” is largely CO with a smaller portion of unreacted CO. The rWGS reaction is moderately endothermic and is thermodynamically favored at higher operating temperatures. Typically, temperatures in the range of about 700° C. to 850° C. are favorable to generate significant amount of CO for Fischer Tropsch and methanol synthesis applications. Only select catalysts may operate at these temperatures and expensive equipment and utilities are needed to heat the streams and system to these temperatures.

Developments in the field of gas fermentation, have resulted in a change in the ratio of gases needed to be provided by a rWGS process for use in gas fermentation applications. Most notable is the departure from the Fischer Tropsch requirement of greater than 90% conversion of CO₂ to CO to a significantly different range of about 20 to about 60% conversion of CO₂ to CO. As used herein, the term “low” used to describe conversion of CO₂ to CO is meant to include from about 20% to about 60% conversion. The syngas produced by a low conversion rWGS has a much different composition from that described above. With low conversion rWGS, the resulting syngas is not largely CO with relatively little CO, but instead may be largely unreacted CO₂ with some, equal parts, or slightly more generated CO. Multiple engineering, operational, and cost advantages arise from the new effluent requirement of a rWGS process. Of particular note is the change in operating temperature of the rWGS unit when the conversion requirements are low. The operating temperature is similarly lowered and the rWGS unit may be operated at temperatures of about 300° C., 325° C., 350° C., 400° C., 425° C., 450° C., 475° C., 500° C., 525° C., 550° C., or 600° C. Suitable ranges of temperatures include 300° C. to 600° C., 400° C. to 550° C., 425° C. to 525° C., and 450° C. to 500° C., 310° C. to 450° C., 310° C. to 425° C., 300° C. to 450° C. At the lower temperatures, the rWGS process may be heated using electric heaters instead of the required fired heaters necessary to operate the rWGS process for Fisher Tropsch applications. Electric heaters are far less costly than fired heaters, and more importantly, consume less energy for operation. Furthermore, with lower operating temperatures, the rWGS process may not need to have multiple reactor stages with interstage heating, and instead could be designed as a single stage at low temperature thus eliminating inter stage heating. Similarly, the need for tubular reactors often employed in high temperature applications is eliminated. One simple adiabatic fixed bed reactor may be sufficient. Thermodynamics indicate that a rWGS process using a simple adiabatic fixed bed reactor with a reactor inlet temperature of 600° C. can provide 35% CO₂ conversion at a reactor exit temperature of about 475° C. The simple adiabatic fixed bed reactor may have a single bed of catalyst. The adiabatic fixed bed reactor may be operated in a once-through mode of operation with no interstage heating. This is a significant improvement over traditional rWGS processes which tend to employ a low temperature reactor, but in combination with a high temperature reactor. The present disclosure allows for the elimination of the high temperature rWGS reactor.

Furthermore, a greater selection of catalysts may be employed due to the lower operating temperatures. Due to thermal stability and high oxygen mobility, ironbased catalysts are often considered as one of the most successful class of catalysts for higher temperatures. Such catalysts include Fe/Al₂O₃, Fe—Cu/Al₂O₃, Fe—Cs/Al₂O₃, Fe—Cu—Cs/Al₂O₃. For lower temperature range operation, copper or copper oxides are often regarded to be successful due to its enhanced adsorption of reaction intermediates by the catalyst support at these lower temperatures. The most common supports are alumina or alumina with zinc oxide. Noble metals such as platinum have also been used. A sample catalyst composition includes 32-335 CuO, 34-53% ZnO and 15-33% Al₂O₃.

Of course, in an alternative embodiment, a high temperature rWGS unit may still be employed with operation altered so as to only meet the lower conversion requirements as compared to typical industrial uses today. For example, the rWGS unit maybe smaller in size, operated at a higher space velocity, or operating on only a portion of the ultimate feed stream to the gas fermentation unit.

Another technical effect that is recognized by moving to a low conversion in the rWGS is that the amount of water generated as a byproduct is similarly reduced. Water is known to deactivate many catalysts and reducing the amount of water generated may increase catalyst life. Further, although the water produced may also be employed in the gas fermentation process, logistically the water does not follow the same detailed flows scheme path as the generated syngas. Therefore, the water must be separated from the syngas. In the low conversion rWGS process, since less water is produced, the water separation equipment may be scaled down to match the water production resulting in less capital investment and reduced operating costs. It is envisioned that with less water to be separated, perhaps a greater range of vapor-liquid separation techniques may be suitable for use. Distillation with fewer theoretical plates, and gravity separators such as knock out pots, settlers, vane separators, mist separators, centrifugal separators, cyclone separators, coalescers, may become available.

Yet another technical benefit in the flow scheme employing a low conversion rWGS process in service for gas fermentation is the opportunity for heat integration. Residual heat in the effluent stream from the rWGS process may be heat exchanged with, for example, the feed stream to the rWGS process thereby reducing overall energy and operating costs.

Turning now to the Figure, the disclosure is described in relation to one embodiment but is not meant to limit the scope. Integrated low conversion rWGS and gas fermentation process 100 begins with CO₂ source 105 and hydrogen source 110. CO₂ source 105, which provides CO₂ feed stream 101, may be a waste gas obtained as a byproduct of an industrial process or from another source, such as combustion engine exhaust fumes, biogas, landfill gas, direct air capture, or from electrolysis. In certain embodiments, the industrial process is selected from ferrous metal products manufacturing, such as a steel manufacturing, non-ferrous products manufacturing, petroleum refining, electric power production, carbon black production, paper and pulp manufacturing, ammonia production, methanol production, coke manufacturing, petrochemical production, carbohydrate fermentation, cement making, aerobic digestion, anerobic digestion, catalytic processes, natural gas extraction, cellulosic fermentation, oil extraction, industrial processing of geological reservoirs, processing fossil resources such as natural gas coal and oil, or any combination thereof. Examples of specific processing steps within an industrial process include catalyst regeneration, fluid catalyst cracking, and catalyst regeneration. Air separation and direct air capture are other suitable industrial processes. Specific examples in steel and ferroalloy manufacturing include blast furnace gas, basic oxygen furnace gas, coke oven gas, direct reduction of iron furnace top-gas, and residual gas from smelting iron. Other general examples include flue gas from fired boilers and fired heaters, such as naturel gas, oil, or coal fired boilers or heaters, and gas turbine exhaust. In these embodiments, the CO₂ may be captured from the industrial process before it is emitted into the atmosphere, using any known method. In some cases, the CO₂ source may generate the CO₂ feed stream 101 as a CO₂-rich syngas which may be obtained from reforming, partial oxidation, or gasification processes. Examples of gasification processes include gasification of coal, gasification of refinery residues, gasification of petroleum coke, gasification of biomass, gasification of lignocellulosic material, gasification of waste wood, gasification of black liquor, gasification of municipal solid waste, gasification of municipal liquid waste, gasification of industrial solid waste, gasification of industrial liquid waste, gasification of refuse derived fuel, gasification of sewerage, gasification of sewerage sludge, gasification of sludge from wastewater treatment, gasification of biogas such as when bigas is added to enhance gasification of another material. Examples of reforming processes include, steam methane reforming, steam naphtha reforming, reforming of natural gas, reforming of biogas, reforming of landfill gas, naphtha reforming, and dry methane reforming. Examples of partial oxidation processes include thermal and catalytic partial oxidation processes, catalytic partial oxidation of natural gas, partial oxidation of hydrocarbons. Examples of municipal solid waste include tires, plastics, and fibers such as in shoes, apparel, and textiles. Municipal solid waste may be simply landfill-type waste and may be sorted or unsorted. Examples of biomass may include lignocellulosic material and microbial biomass. Lignocellulosic material may include agriculture waste and forest waste.

Hydrogen source 105 provides hydrogen feed stream 102. Hydrogen source 105 may be hydrogen generated by electrolysis, such as in the electrolysis of water or photo electrolysis. Hydrogen source 105 may be one or more of many sources such as thermochemical, electrolysis, direct solar water splitting, and or biological. Common sources include other readily available process units which produce hydrogen as a byproduct. Reforming examples include steam, partial oxidation, autothermal, plasma, and aqueous phase. Non-fossil sources are particularly advantageous from a sustainability perspective.

CO₂ feed stream 101 and hydrogen feed stream 102 may be combined into a combined CO₂ and hydrogen feed stream 103. Such combination is not required however, and each feed stream 101 and 102 may remain independent up until the preheater 125 discussed below. The option of keeping each feed stream 101 and 102 independent (not shown in Figure) is important when only one of the feed streams require compression and or contaminant removal. In this way only the feed stream requiring compression or contaminant removal need be processed thereby saving in capital and operational costs. For ease of explanation the Figure depicts the embodiment where CO₂ feed stream 101 and hydrogen feed stream 102 are combined into a combined CO₂ and hydrogen feed stream 103.

Combined CO₂ and hydrogen feed stream 103 is passed to compressor 115 to be brought to the desired pressure. Of course, if combined CO₂ and hydrogen feed stream 103 is already at suitable pressure by virtue of CO₂ source 105 and or hydrogen source 110, compressor 115 may not be necessary. Compressor 115 may be a single compressor or may be a set of two or more compressors. Compressor 115 is elected with sufficient specifications to compress feed stream 103 to the desired pressure for rWGS reactor 135. Compressed feed stream 116 provided by compressor 115 is passed to optional first contaminate removal unit 120. First contaminate removal unit 120 is optional and only employed in those circumstances where contaminates are present which may harm the rWGS catalyst or perhaps the gas fermentation biocatalyst. It is understood that if only the CO₂ feed stream 101 or only the hydrogen feed steam 102 contain a contaminate which needs to be removed in optional first removal unit 120, the option of not combining feed streams 101 and 102 into combined stream 103 may be advantageous thereby reducing the size or cost of optional first removal unit 120. Optional first removal unit 120 may be any type of unit suitable to remove the contaminate of concern. For example, optional first removal unit 120 may be an adsorption unit or a catalytic unit which reacts the contaminate to form a non-harmful product which may be processed without fear of deactivation of downstream inorganic or perhaps biological catalysts. Physical removal of the contaminate such as when using an adsorptive technique may result in optional first contaminant stream 123. Compressed clean feed stream 121 is produces as the effluent from optional first removal unit 120

Compressed clean feed stream 121 is passed to pre-heater 125. Pre-heater 125 may be a heat exchanger such as an indirect heat exchanger. Compressed clean feed stream 121 is heat exchanged in pre-heater 125 with rWGS reactor effluent 136 to capture and employ heat remaining in rWGS reactor effluent 136. Heat integration minimizes energy loss and redeploys exiting heat where it is needed resulting in overall reduction in energy costs. Partially heated compressed clean feed stream 126 is passed to electric heater 130 to bring the temperature of the stream to that needed for the rWGS reaction including adjustments in the operating temperature of the rWGS reactor 135 for the desired pre-determined percent conversion of CO₂ to CO. Employing electric heater 130 is a significant advancement over previous flow schemes since requirement for a fired heater has been eliminated. Electric heater 130 provides economic and environmental benefits over a fired heater, including the elimination of combustion emissions which provides an enhanced degree of sustainability.

Fully heated compressed clean feed stream 131 from electric heater 130 is passed to rWGS reactor 135. As discussed above, rWGS reactor 135 is advantageously operated at low temperature as compared to those used in service of Fischer Tropsch or methanol synthesis processes. rWGS reactor 135 is a low conversion rWGS reactor as discussed above. Suitable catalysts and operating conditions for the rWGS reactor 135 as well as reactor types and modes of operation are discussed above. rWGS reactor effluent 136 contains the generated syngas at the desired CO concentration. rWGS reactor effluent 136 is heat exchanged in pre-heater 125 to recover residual heat as discussed above, resulting in partially cooled rWGS reactor effluent 137. An exemplary clean feed stream 131 may contain a H₂:CO₂ molar ratio of about 3:1. An exemplary rWGS effluent 136 may contain a H₂:CO:CO₂ molar ratio of about 5:1:1. An exemplary rWGS effluent 136 may be at a pressure ranging from about 5 to about 8 barg. Another exemplary rWGS effluent 136 may be at a pressure ranging from about 3 to about 4 barg. An exemplary rWGS effluent 136 may be at or near to (+/− about 5 degrees C.) ambient temperature. Another exemplary rWGS effluent 136 may be saturated with water.

Water is produced by the rWGS reaction and at least a portion of the water present in partially cooled rWGS reactor effluent 137 should be removed. Therefore, partially cooled rWGS reactor effluent 137 is passed to air cooler 140 to reduce the temperature of partially cooled rWGS reactor effluent 137 further and generate fully cooled rWGS reactor effluent 141. The reduction in temperature serves two purposes. Firstly, the separation of water from syngas is more readily accomplished at lower temperatures and secondly, gas fermentation process 155 operates at temperatures far lower than needed for low conversion rWGS. Fully cooled rWGS reactor effluent is passed to water knock out 145 to separate water stream 147 from syngas stream 146. Water stream 147 may be further integrated in the overall process by being routed to gas fermentation process 155 and used within the gas fermentation process 155 (integration not shown in Figure).

Commercially important gas fermentation processes often employ anerobic biocatalysts. Therefore, syngas stream 146 may be passed to optional second containment removal zone 150 for the removal of oxygen. Other contaminates may also be present as a result of the rWGS operation which may need to be removed in second contaminate removal zone 150. In the embodiment where gas fermentation process 155 employs an anerobic biocatalyst, oxygen stream 152 is removed from second contaminate removal zone 150. Further integration may be possible through using oxygen stream 152 elsewhere in the overall process. For example, oxygen stream 152 maybe employed in a gasifier that part of the CO₂ source 105. Other contaminants removed in optional second containment removal zone 150 include microbe inhibitors and/or biocatalyst inhibitors that may be found in syngas stream 146. Such contaminants may include, but are not limited to, sulphur compounds, aromatic compounds, alkynes, alkenes, alkanes, olefins, nitrogen compounds, phosphorous-containing compounds, particulate matter, solids, oxygen, halogenated compounds, silicon-containing compounds, carbonyls, metals, alcohols, esters, ketones, peroxides, aldehydes, ethers, and tars.

Clean syngas stream 151 is passed to gas fermentation process 155 which is a metabolic process that produces chemical changes in the CO and CO₂ components of clean syngas stream 151. Gas fermentation process 155 employs one or more C1-fixing microorganisms as biocatalyst. Gas fermentation process 155 may include one or bioreactors and may be operated in batch mode or continuous mode. In batch fermentation the bioreactor is filled with raw material carbon source, along with microorganism biocatalyst(s), and the products remain in the bioreactor until fermentation is completed whereupon the products are extracted, and the bioreactor is cleaned before the next “batch” is started. In continuous fermentation, the fermentation process is extended for longer periods of time, and product and/or metabolite is extracted during fermentation. In one embodiment, the fermentation process is operated in a continuous mode. Suitable biocatalysts for use in gas fermentation process 155 are known and are know discussed here in detail. Further details may be found in the following references. The microorganism employed as the biocatalyst of gas fermentation process 155 may produce or may be engineered to produce ethanol (WO 2007/117157), acetate (WO 2007/117157), 1-butanol (WO 2008/115080, WO 2012/053905, and WO 2017/066498), butyrate (WO 2008/115080), 2,3-butanediol (WO 2009/151342 and WO 2016/094334), lactate (WO 2011/112103), butene (WO 2012/024522), butadiene (WO 2012/024522), methyl ethyl ketone (2-butanone) (WO 2012/024522 and WO 2013/185123), ethylene (WO 2012/026833), acetone (WO 2012/115527), isopropanol (WO 2012/115527), lipids (WO 2013/036147), 3-hydroxypropionate (3-HP) (WO 2013/180581), terpenes, including isoprene (WO 2013/180584), fatty acids (WO 2013/191567), 2-butanol (WO 2013/185123), 1,2-propanediol (WO 2014/036152), 1-propanol (WO 2017/066498), 1-hexanol (WO 2017/066498), 1-octanol (WO 2017/066498), chorismate-derived products (WO 2016/191625), 3-hydroxybutyrate (WO 2017/066498), 1,3-butanediol (WO 2017/066498), 2-hydroxyisobutyrate or 2-hydroxyisobutyric acid (WO 2017/066498), isobutylene (WO 2017/066498), adipic acid (WO 2017/066498), 1,3-hexanediol (WO 2017/066498), 3-methyl-2-butanol (WO 2017/066498), 2-buten-1-ol (WO 2017/066498), isovalerate (WO 2017/066498), isoamyl alcohol (WO 2017/066498), and monoethylene glycol (WO 2019/126400). In specific embodiments the biocatalyst are C1-fixing microorganisms that have the ability to produce one or more products from a C1 carbon source. Suitable microorganism may be a member of a genus selected from the group consisting of Acetobacterium, Alkalibaculum, Blautia, Butyribacterium, Clostridium, Eubacterium, Moorella, Oxobacter, Sporomusa, and Thermoanaerobacter. In particular, the microorganism may be derived from a parental bacterium selected from the group consisting of Acetobacterium woodii, Alkalibaculum bacchii, Blautia producta, Butyribacterium methylotrophicum, Clostridium aceticum, Clostridium autoethanogenum, Clostridium carboxidivorans, Clostridium coskatii, Clostridium drakei, Clostridium formicoaceticum, Clostridium ljungdahlii, Clostridium magnum, Clostridium ragsdalei, Clostridium scatologenes, Eubacterium limosum, Moorella thermautotrophica, Moorella thermoacetica, Oxobacter pfennigii, Sporomusa ovata, Sporomusa silvacetica, Sporomusa sphaeroides, and Thermoanaerobacter kivui. In a specific embodiment, the microorganism is derived from the cluster of Clostridia comprising the species Clostridium autoethanogenum, Clostridium ljungdahlii, and Clostridium ragsdalei.

Clean syngas stream 151 comprises the desired targeted amount of CO, such as about 1, 2, 5, 10, 20, 30, 40, 50, 60, 70, 80, or 90 mol % CO. Clean syngas stream 151 may comprise a range of CO, such as about 20-80, 30-70, or 40-60 mol % CO. In some embodiments, clean syngas stream 151 may comprise a relatively low amount of CO, such as about 1-10 or 1-20 mol-% CO. The microorganism biocatalyst typically converts at least a portion of the CO in the substrate to a product. In some embodiments, clean syngas stream 151 comprises no or substantially no (<1 mol %) CO. Clean syngas stream 151 further comprises some amount of hydrogen. For example, clean syngas stream 151 may comprise about 1, 2, 5, 10, 15, 20, or 30 mol-% hydrogen. In some embodiments, clean syngas stream 151 may comprise a relatively high amount of hydrogen, such as about 60, 70, 80, or 90 mol % hydrogen. In further embodiments, clean syngas stream 151 comprises no or substantially no (<1 mol-%) hydrogen. Clean syngas stream 151 further comprises some amount of CO₂. For example, clean syngas stream 151 may comprise about 1-80, 20-80, 20-60, 40-60 or 1-30 mol % CO₂.

Gas fermentation process 155 employs one or more bioreactors which include a culture/fermentation device consisting of one or more vessels, towers, or piping arrangements, such as a continuous stirred tank reactor (CSTR), immobilized cell reactor (ICR), trickle bed reactor (TBR), bubble column, gas lift fermenter, static mixer, or other vessel or other device suitable for gas-liquid contact. In some embodiments, the bioreactor may comprise a first growth reactor and a second culture/fermentation reactor. The substrate may be provided to one or both of these reactors. As used herein, the terms “culture” and “fermentation” are used interchangeably. These terms encompass both the growth phase and product biosynthesis phase of the culture/fermentation process.

The culture is generally maintained in an aqueous culture medium that contains nutrients, vitamins, and/or minerals sufficient to permit growth of the microorganism. Preferably the aqueous culture medium is an anaerobic microbial growth medium, such as a minimal anaerobic microbial growth medium. Suitable media are well known in the art.

The culture/fermentation should desirably be carried out under appropriate conditions for production of the target product. Typically, the culture/fermentation is performed under anaerobic conditions. Reaction conditions to consider include pressure (or partial pressure), temperature, gas flow rate, liquid flow rate, media pH, media redox potential, agitation rate (if using a continuous stirred tank reactor), inoculum level, maximum gas substrate concentrations to ensure that gas in the liquid phase does not become limiting, and maximum product concentrations to avoid product inhibition. In particular, the rate of introduction of the substrate may be controlled to ensure that the concentration of gas in the liquid phase does not become limiting, since products may be consumed by the culture under gas-limited conditions.

Operating a bioreactor at elevated pressures allows for an increased rate of gas mass transfer from the gas phase to the liquid phase. Accordingly, it is generally preferable to perform the culture/fermentation at pressures higher than atmospheric pressure. Also, since a given gas conversion rate is, in part, a function of the substrate retention time and retention time dictates the required volume of a bioreactor, the use of pressurized systems can greatly reduce the volume of the bioreactor required and, consequently, the capital cost of the culture/fermentation equipment. This, in turn, means that the retention time, defined as the liquid volume in the bioreactor divided by the input gas flow rate, can be reduced when bioreactors are maintained at elevated pressure rather than atmospheric pressure. The optimum reaction conditions will depend partly on the particular microorganism used. However, in general, it is preferable to operate the fermentation at a pressure higher than atmospheric pressure. Also, since a given gas conversion rate is in part a function of substrate retention time and achieving a desired retention time in turn dictates the required volume of a bioreactor, the use of pressurized systems can greatly reduce the volume of the bioreactor required, and consequently the capital cost of the fermentation equipment.

In certain embodiments, the fermentation is performed in the absence of light or in the presence of an amount of light insufficient to meet the energetic requirements of photosynthetic microorganisms. In certain embodiments, the microorganism of the invention is a non-photosynthetic microorganism.

As used herein, the terms “fermentation broth” or “broth” refer to the mixture of components in a bioreactor, which includes cells and nutrient media. As used herein, a “separator” is a module that is adapted to receive fermentation broth from a bioreactor and pass the broth through a filter to yield a “retentate” and a “permeate.” The filter may be a membrane, e.g. a cross-flow membrane or a hollow fibre membrane. The term “permeate” is used to refer to substantially soluble components of the broth that pass through the separator. The permeate will typically contain soluble fermentation products, byproducts, and nutrients. The retentate will typically contain cells. As used herein, the term “broth bleed” is used to refer to a portion of the fermentation broth that is removed from a bioreactor and not passed to a separator.

Target products may be separated or purified from a fermentation broth using any method or combination of methods known in the art, including, for example, fractional distillation, evaporation, pervaporation, gas stripping, phase separation, and extractive fermentation, including for example, liquid-liquid extraction. In certain embodiments, target products are recovered from the fermentation broth by continuously removing a portion of the broth from the bioreactor, separating microbial cells from the broth (conveniently by filtration), and recovering one or more target products from the broth. Alcohols and/or acetone may be recovered, for example, by distillation. Acids may be recovered, for example, by adsorption on activated charcoal. Separated microbial cells are preferably recycled back to the bioreactor. The cell-free permeate remaining after target products have been removed is also preferably returned to the bioreactor. Additional nutrients may be added to the cell-free permeate to replenish the medium before it is returned to the bioreactor. Desired products are removed from gas fermentation process 155 in gas fermentation product stream 158.

The bioreactors of gas fermentation process 155 also produce bioreactor off gas stream 156 which contains unreacted C1 gases and inert gases. If gas fermentation process 155 employs inoculator reactor(s) as well, gas fermentation process 155 may also produce inoculator off gas stream 157 which similarly contains unreacted C1 gases and inert gases. Bioreactor off-gas stream 156 and optional inoculator off-as stream 157 are passed to recycle compressor 160 and compressed recycle stream 161 is passed back to gas fermentation process 155.

The desired products from the gas fermentation process may be passed to a downstream processing system to generate yet another product. The downstream processing system may be selected from an olefins plant, a poly-olefins plant, a polypropylene plant, a polyethylene plant, a polymer plant, a high-density polyethylene plant, an oligomers plant, a nitriles plant, an oxides plant, a topping refinery, a hydro-skimming refinery, a conversion refinery, a deep conversion refinery, a coking unit, a stream cracker, or a styrenics plant.

All references, including publications, patent applications, and patents, cited herein are hereby incorporated by reference to the same extent as if each reference were individually and specifically indicated to be incorporated by reference and were set forth in its entirety herein. The reference to any prior art in this specification is not, and should not be taken as, an acknowledgement that that prior art forms part of the common general knowledge in the field of endeavor in any country.

The use of the terms “a” and “an” and “the” and similar terms are to be construed to cover both the singular and the plural, unless otherwise indicated herein or clearly contradicted by context. The terms “comprising,” “having,” “including,” and “containing” are to be construed as open-ended terms unless otherwise noted. The use of the alternative, such as the term “or”, should be understood to mean either one, both, or any combination thereof of the alternatives. As used herein, the term “about” means±20% of the indicated range, value, or structure, unless otherwise indicated.

Recitation of ranges of values herein are merely intended to serve as a shorthand method of referring individually to each separate value falling within the range, unless otherwise indicated herein, and each separate value is incorporated into the specification as if it were individually recited herein. For example, any concentration range, percentage range, ratio range, integer range, size range, or thickness range is to be understood to include the value of any integer within the recited range and, when appropriate, fractions thereof (such as one tenth and one hundredth of an integer), unless otherwise indicated.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

An example 1 is a process for producing a product by gas fermentation comprising
a. passing a combination feed stream comprising CO₂ and hydrogen to a preheater wherein the combination feed stream comprises a first feed stream comprising CO₂ and a second feed stream comprising hydrogen;
b. heat exchanging the combination feed stream with a reverse water gas shift reactor effluent stream to partially heat the combination feed stream and partially cool the reverse water gas shift reactor effluent stream;
c. passing the partially heated combination feed stream to an electric heater to fully heat the combination feed stream to a predetermined inlet temperature of a low conversion reverse water gas shift reactor;
d. passing the fully heated combination feed stream to the low conversion reverse water gas shift reactor to generate the reverse water gas shift reactor effluent stream comprising CO, CO₂, hydrogen, and water, wherein the conversion of CO₂ to CO in the low conversion reverse water gas shift reactor is from about 20 to about 60 mass percent;
e. passing the partially cooled reverse water gas shift reactor effluent stream to a cooler to generate a fully cooled reverse water gas shift reactor effluent stream;
f. removing water from the fully cooled reverse water gas shift reactor effluent stream and removing at least one biocatalyst inhibitor to generate a syngas feed stream; and
g. passing the syngas feed stream to a gas fermentation process to generate a gas fermentation product stream and a gas fermentation off gas stream comprising unreacted reactants selected from CO, CO₂, and hydrogen.

The process of the preceding example 1 further comprising recycling the gas fermentation off gas stream to the gas fermentation process.

The process of any preceding example wherein the low conversion reverse water gas shift reactor is operated at a temperature less than about 600° C., less than about 500° C., less than about 400° C., less than about 350° C., less than about 310° C. or less than about 300° C.

The process of any preceding example wherein the low conversion reverse water gas shift reactor employs a catalyst comprising copper or copper oxide.

The process of any preceding example further comprising compressing the first feed stream comprising CO₂, the second feed stream comprising hydrogen, and or the combination feed stream comprising CO₂ and hydrogen.

The process of any preceding example further comprising removing one or more contaminates from the first feed stream comprising CO₂, the second feed stream comprising hydrogen, and or the combination feed stream comprising CO₂ and hydrogen.

The process of any preceding example wherein the syngas feed stream is at a pressure ranging from about 5 to about 8 barg.

The process of any preceding example, wherein the syngas feed stream is at a pressure ranging from about 3 to about 4 barg.

The process of any preceding example, wherein the syngas feed stream comprises a molar ratio of H₂:CO:CO₂ of about 5:1:1.

The process of any preceding example, wherein the combination feed stream comprises a molar ratio of H₂:CO₂ of from about 3:1.

The process of any preceding example, wherein the syngas feed stream comprises a molar ratio of H₂:CO:CO₂ of about 5:1:1 and the combination feed stream comprises a molar ratio of H₂:CO₂ of from about 3:1.

The process of any preceding example, wherein the low conversion reverse water gas shift reactor is a fixed bed adiabatic reactor.

The process of any preceding example, wherein the fixed bed adiabatic comprises a single bed of catalyst.

The process of any preceding example, wherein the low conversion reverse water gas shift reactor is operated in a once through mode of operation with no interstage heating.

The process of any preceding example wherein the gas fermentation product stream is passed to a downstream processing system to generate another product.

The process of any preceding example wherein the downstream processing system is selected from an olefins plant, a poly-olefins plant, a polypropylene plant, a polyethylene plant, a polymer plant, a high-density polyethylene plant, an oligomers plant, a nitriles plant, an oxides plant, a topping refinery, a hydro-skimming refinery, a conversion refinery, a deep conversion refinery, a coking unit, a stream cracker, or a styrenics plant.

The process of any preceding example, wherein the gas fermentation process is a microbial fermentation process employing a C1-fixing microorganism in a nutrient solution

The process of any preceding example wherein the C1 fixing microorganism is aerobic or anaerobic.

The process of any preceding example, wherein the C1-fixing microorganism is selected from a genus of Clostridium, Moorella, Carboxydothermus, Ruminococcus, Acetobacterium, Eubacterium, Butyribacterium, Oxobacter, Methanosarcina, Methanosarcina, Desulfotomaculum, and Cupriavidus.

The process of any preceding example, wherein the gas fermentation product stream comprises at least one product selected from ethylene, ethanol, propane, acetate, 1-butanol, butyrate, 2,3-butanediol, lactate, butene, butadiene, methyl ethyl ketone (2-butanone), acetone, isopropanol, a lipid, 3-hydroxypropionate (3-HP), a terpene, isoprene, a fatty acid, 2-butanol, 1,2-propanediol, 1propanol, 1hexanol, 1octanol, chorismate-derived products, 3hydroxybutyrate, 1,3butanediol, 2-hydroxyisobutyrate or 2-hydroxyisobutyric acid, isobutylene, adipic acid, 1,3hexanediol, 3-methyl-2-butanol, 2-buten-1-ol, isovalerate, isoamyl alcohol, and monoethylene glycol, or any combination thereof.

1. A process for producing a product by gas fermentation comprising
a. passing a combination feed stream comprising CO₂ and hydrogen to a preheater wherein the combination feed stream comprises a first feed stream comprising CO₂ and a second feed stream comprising hydrogen;
b. heat exchanging the combination feed stream with a reverse water gas shift reactor effluent stream to partially heat the combination feed stream and partially cool the reverse water gas shift reactor effluent stream;
c. passing the partially heated combination feed stream to an electric heater to fully heat the combination feed stream to a predetermined inlet temperature of a low conversion reverse water gas shift reactor;
d. passing the fully heated combination feed stream to the low conversion reverse water gas shift reactor to generate the reverse water gas shift reactor effluent stream comprising CO, CO₂, hydrogen, and water, wherein the conversion of CO₂ to CO in the low conversion reverse water gas shift reactor is from about 20 to about 60 mass percent;
e. passing the partially cooled reverse water gas shift reactor effluent stream to a cooler to generate a fully cooled reverse water gas shift reactor effluent stream;
f. removing water from the fully cooled reverse water gas shift reactor effluent stream and removing at least one biocatalyst inhibitor to generate a syngas feed stream; and
g. passing the syngas feed stream to a gas fermentation process to generate a gas fermentation product stream and a gas fermentation off gas stream comprising unreacted reactants selected from CO, CO₂, and hydrogen.

2. The process of claim 1 further comprising recycling the gas fermentation off gas stream to the gas fermentation process.

3. The process of claim 1 wherein the low conversion reverse water gas shift reactor is operated at a temperature less than about 600° C., less than about 500° C., less than about 400° C., less than about 350° C., less than about 310° C. or less than about 300° C.

4. The process of claim 3 wherein the low conversion reverse water gas shift reactor employs a catalyst comprising copper or copper oxide.

5. The process of claim 1 further comprising compressing the first feed stream comprising CO₂, the second feed stream comprising hydrogen, and or the combination feed stream comprising CO₂ and hydrogen.

6. The process of claim 1 further comprising removing one or more contaminates from the first feed stream comprising CO₂, the second feed stream comprising hydrogen, and or the combination feed stream comprising CO₂ and hydrogen.

7. The process of claim 1 wherein the syngas feed stream is at a pressure ranging from about 5 to about 8 barg.

8. The process of claim 1 wherein the syngas feed stream is at a pressure ranging from about 3 to about 4 barg.

9. The process of claim 1 wherein the syngas feed stream comprises a molar ratio of H₂:CO:CO₂ of about 5:1:1.

10. The process of claim 1 wherein the combination feed stream comprises a molar ratio of H₂:CO₂ of from about 3:1.

11. The process of claim 1 wherein the syngas feed stream comprises a molar ratio of H₂:CO:CO₂ of about 5:1:1 and the combination feed stream comprises a molar ratio of H₂:CO₂ of from about 3:1.

12. The process of claim 1 wherein the low conversion reverse water gas shift reactor is a fixed bed adiabatic reactor.

13. The process of claim 12 wherein the fixed bed adiabatic comprises a single bed of catalyst.

14. The process of claim 1 wherein the low conversion reverse water gas shift reactor is operated in a once through mode of operation with no interstage heating.

15. The process of claim 1 wherein the gas fermentation product stream is passed to a downstream processing system to generate another product.

16. The process of claim 15 wherein the downstream processing system is selected from an olefins plant, a poly-olefins plant, a polypropylene plant, a polyethylene plant, a polymer plant, a high-density polyethylene plant, an oligomers plant, a nitriles plant, an oxides plant, a topping refinery, a hydro-skimming refinery, a conversion refinery, a deep conversion refinery, a coking unit, a stream cracker, or a styrenics plant.

17. The process of claim 1 wherein the gas fermentation process is a microbial fermentation process employing a C1-fixing microorganism in a nutrient solution.

18. The process of claim 16 wherein the C1 fixing microorganism is aerobic or anaerobic.

19. The process of claim 16 wherein the C1-fixing microorganism is selected from a genus of Clostridium, Moorella, Carboxydothermus, Ruminococcus, Acetobacterium, Eubacterium, Butyribacterium, Oxobacter, Methanosarcina, Methanosarcina, Desulfotomaculum, and Cupriavidus.

20. The process of claim 16 wherein the gas fermentation product stream comprises at least one product selected from ethylene, ethanol, propane, acetate, 1-butanol, butyrate, 2,3-butanediol, lactate, butene, butadiene, methyl ethyl ketone (2-butanone), acetone, isopropanol, a lipid, 3-hydroxypropionate (3-HP), a terpene, isoprene, a fatty acid, 2-butanol, 1,2-propanediol, 1propanol, 1hexanol, 1octanol, chorismate-derived products, 3hydroxybutyrate, 1,3butanediol, 2-hydroxyisobutyrate or 2-hydroxyisobutyric acid, isobutylene, adipic acid, 1,3hexanediol, 3-methyl-2-butanol, 2-buten-1-ol, isovalerate, isoamyl alcohol, and monoethylene glycol, or any combination thereof.
